(19) United States
(12) Patent Application Publication
(10) Pub. No.: US 2012/0321730 A1
(43) Pub. Date: Dec. 20, 2012

(54) CAPSICUM ANNUUM EXTRACT AND PROCESS THEREOF

(75) Inventors: Chennakattu Varkey Jacob, Kerala (IN); Sanjith Madhavan, Kerala (IN); Shaji Kannanaikkal Jacob, Kerala (IN); Rekha Ramachandra Mallia, Kerala (IN)

(73) Assignee: SYNTHITE INDUSTRIES LTD., Kerala (IN)

(21) Appl. No.: 13/581,609

(22) PCT Filed: Apr. 27, 2011
(86) PCT No.: PCT/IB2011/051826

§ 371 Date: Aug. 28, 2012

(30) Foreign Application Priority Data
Apr. 29, 2010 (IN) 1205/CHE/2010

(51) Int. Cl. A61K 36/81; A61P 3/00; A61K 8/97; A23L 1/48; C11B 5/00

(52) U.S. Cl. 424/760; 426/655; 426/542

(57) ABSTRACT

The present disclosure provides an industrial process for isolation and purification of capsanthin rich carotenoid mixture from paprika oleoresin extracted from Capsicum annuum. The process yields a water dispensable powder product with high pigment value and an extended shelf life. With its high antioxidant values and HDL-cholesterol-raising effect, the carotenoid mixture can be used as a nutraceutical supplement and as a food additive.

### TECHNICAL FIELD

The present disclosure relates to a method of isolation and purification of Carotenoids rich in trans-capsanthin content from Capsicum annuum. The Capsicum annuum extract comprises Carotenoids in a range of about 50% w/w to about 80% w/w. The extract can be utilized to enhance HDL cholesterol in plasma, for cosmetic applications, as nutraceutical, as antioxidant and as health supplement.

### BACKGROUND

Carotenoids are commercially used as food colorants and nutritional supplements. Hundreds of Carotenoids have been isolated from higher plants, of which lycopene, lutein and zeaxanthin are regarded as nutraceutical because of their physiological effects. Carotenoids which are naturally found in plants, algae, bacteria and fungi are regarded as one of the most beneficial compounds for cancer prevention in humans.

Dietary carotenoids accumulate in many tissues, including the liver, adipose tissue, serum, breast milk, adrenal, prostate, macula, kidney, lung, brain and skin. Dermal accumulation of carotenoids is related to the ability of the carotenoids to act as scavengers of singlet oxygen and peroxyl radicals and thus potentially provide protection from UV radiation. Also increased levels of lutein and zeaxanthin in the macula of the eye have been associated with decreased risk of age-related macular degeneration, the leading cause of blindness in the elderly.

Trans-capsanthin, cis-capsanthin, capsanthin-5,6-epoxides and capsorubin are main carotenoids found exclusively in red fruits of capsicum spp, and other significant carotenoids—lutein, zeaxanthin, violaxanthin, anthreraxanthin, β-cryptoxanthin and β-carotene that act as the precursors of the former, are found in the yellow and orange colour. Capsanthin, uniquely found in capsicum species, is a fat soluble red pigment. Capsaicin is the major flavouring compound, whereas capsanthin and capsorubin are major colouring compounds among variety of coloured compounds present in paprika oleoresin.

Capsanthin is synthesized during carotenogenesis and enhances liposolubility by esterifying with short-chain saturated fatty acids. The absorptive pathways of carotenoids are similar to those of other dietary lipids. Capsanthin is found abundantly distributed at the polar surfaces of lipoproteins, and the clearance of capsanthin is much faster than those of lycopene in the human body. The bioavailability of carotenoids other than β-carotene in humans should be clarified to assess the anticarcinogenic effect of dietary carotenoids.

Biological antioxidants can be grouped into two; preventive antioxidants which reduce the initiation of lipid peroxidation by suppressing the generation of chain-initiating radicals, and; chain-breaking antioxidants which interfere with chain propagation by trapping the chain-initiating or chain-propagating peroxy radicals. Carotenoids are generally classified as preventive antioxidants, because they deactivate the singlet oxygen responsible for the formation of lipid hydroperoxides thereby suppressing hydroperoxide-dependent lipid peroxidation. They also can act as chain-breaking antioxidants in certain cases.

Capsanthin, like other xanthophylls, is also regarded as a functional material by antioxidative activity and anti-tumor-promotion activity, even though it exhibit no provitamin A activity, in contrast to β-carotene. However, capsanthin is found to be more effective in the photooxidation of linoleic acid and polyunsaturated fatty acids when compared to β-carotene, lycopene and lutein. This is because, though they possess the same number of conjugated double bonds, in capsanthin the conjugation extends to a keto group and the cyclopentane ring. The oxo group of capsanthin is considered to inhibit the radical-driven hydroperoxidation or methyl linoleate thus exhibiting the antioxidative activity.

Interestingly, capsanthin decomposed more slowly than the other carotenoids, and the radical scavenging effect of capsanthin is found to last longer. It is first found that esterified (monoesterified and diesterified) capsanthin also were good radical scavengers. Also, the capsanthin esterified partially with fatty acids (mono- and/or diesterified capsanthin) isolated from paprika color suppressed oxidation of methyl linoleate in a similar manner as nonesterified capsanthin. These finding suggests that the radical scavenging ability of capsanthin is not influenced by esterification, that is, the anti-oxidant ability would contribute to the polyene chain having conjugated keto group.

Xanthophylls, including capsanthin, are distributed to HDL in larger amounts than to LDL compared with hydrocarbon carotenoids. Xanthophylls being polar carotenoids are likely to be localized at the polar surface of lipoprotein consisting of phospholipids and apoprotein. HDL is rich in phospholipid and apoprotein and therefore may be liable to accumulate xanthophylls. Xanthophylls can act as antioxidants against free radical attack and exposure to singlet oxygen in plasma lipoproteins (Boey et al. 1992, Ojima et al. 1993). Thus, dietary xanthophylls seem to participate in the primary defense of HDL against oxidative attack in the bloodstream and arterial wall. Capsanthin and capsorubin are found to efficiently suppress LDL oxidation, inhibit the formation of conjugated dienes from polyunsaturated fatty acid residues, lower the content of small dense LDL subfractions, and inhibit the transformation of cholesterol to auto oxidized form. Intake of capsanthin may be helpful for increasing the antioxidant defense of this plasma lipoprotein.

Capsanthin is known to have HDL-cholesterol-raising effect on plasma and the potential to increase cholesterol efflux to HDL particles [Koichi Aizawa and Takahiro Inakuma, British Journal of Nutrition (2009), 102, 1760-1766)].

Dietary carotenoids are of much interest in its role in the prevention of degenerative diseases such as cancer and cardiovascular disease (Gaziano and Hennekens 1993, Peto et al. 1981, van Poppel 1993). Carotenoid-rich diet is clearly related to the reduction in the risk of different chronic disorders, such as cardiovascular disease, some type of cancer, and age-related macular degeneration. Epidemiological studies suggest that the incidence of human cancer is inversely correlated with the dietary intake of carotenoids and their concentration in blood plasma (Ziegler 1988).

In epidemiological studies, capsanthin appears to possibly exert a potent inhibitory effect on colon carcinogenesis. It has been reported that capsanthin and capsanthin rich paprika juice might affect colon carcinogenesis via the suppression of aberrant crypt foci formation (Narisawa et al 2000). The study relating to the protective effect of capsicum annuum extracts on carbon tetrachloride-induced hepatotoxicity revealed that capsanthin and β-cryptoxanthin significantly preserved the activity of superoxide dismutase in CCl₄-treated rats, and also strongly inhibit lipid peroxidation, as demonstrated by a reduction in the production of malondialdehyde. Therefore, increased intake of capsanthin rich foods may be helpful for the improvement of health.

A variety of carotenoids are present in commonly eaten foods and these compounds accumulate in tissues and blood plasma. About 20 carotenoids, including their metabolites, have been identified in human plasma (Khachick et al. 1995). Animal studies and cultured cell studies have shown that some carotenoids other than β-carotene possess antitumor properties that are equivalent or superior in effectiveness to β-carotene (Levy et al. 1995, Mura koshi et al. 1992, Pung et al. 1988, Tanaka et al. 1994).

Capsanthin and related carotenoids showed potent in-vitro anti-tumor-promotion effects, with inhibitory effects on Epstein-Barr virus early antigen (EBV-EA) activation induced by tumor promoter TPA, and especially, capsanthin esterified with fatty acids exhibited potent anti-tumor promoting activity in an in-vivo mouse skin two-stage carcinogenesis assay. Dietary capsanthin is absorbed and accumulated in appreciable amounts in human plasma lipoproteins. The distribution of capsanthin in each lipoprotein fraction and clearance rate from plasma were different from those of lycopene, a typical hydrocarbon carotenoid.

Capsicum annuum extract so far reported has only total carotenoids of about 25-30% w/w with capsanthin content in a range of about 30% w/w to about 40% w/w of total carotenoids. The color value reported is in the range of 1-6 lakhs colour unit and red pigment value is about 60 units. The product reported so far is sensitive to heat and light and degrades fast.

In conclusion, there remains a need for a simple and compliant method of the isolation of free xanthophylls, with high carotenoids content, which again is rich in trans-capsanthin. The present disclosure provides a process with improved quality and stability of the carotenoids mixture carried out on a manufacturing scale. The Capsicum annuum extract comprises Carotenoids in a range of about 50% w/w to about 80% w/w, wherein the extract comprises Carotenoid Capsanthin in a range of about 35% w/w to about 60% w/w and the Carotenoids comprises Capsanthin in a range of about 50% w/w to about 80% w/w of Carotenoids. The extract can be utilized to enhance HDL cholesterol in plasma, for cosmetic applications, as nutraceutical, as antioxidant and as health supplement

### STATEMENT OF DISCLOSURE

The present disclosure provides a capsicum annuum extract comprising Carotenoids in a range of about 50% w/w to about 80% w/w; a process for preparing Capsicum annuum extract comprising Carotenoids of about 50% w/w to about 80% w/w, said process comprising acts of, a) subjecting purified paprika to supercritical fluid extraction to obtain deodorized paprika; b) saponifying the deodorized paprika to obtain saponified products; c) dissolving the saponified products in a solvent mixture comprising a organic solvent and water and separating the organic solvent layer and water layer and; d) washing the organic solvent layer with water to neutralize to about pH 7 to obtain the Capsicum annuum extract comprising Carotenoids of about 50% w/w to about 80% w/w and a composition comprising Capsicum annuum extract comprising carotenoids of about of about 50% w/w to about 80% w/w along with one or more excipient.

### BRIEF DESCRIPTION OF ACCOMPANYING FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure where,

FIG. 1 shows color value of carotenoids mixture;

FIG. 2 shows UV spectrum of total carotenoids;

FIG. 3 shows HPLC of trans-Capsanthin;

FIG. 4 shows Color value of Capsanthin from Chinese paprika; and

FIG. 5 shows HPLC of Capsanthin from Chinese paprika.

FIG. 6 shows schematic diagram of the process for obtaining Capsicum annuum extract.

### DETAILED DESCRIPTION OF DISCLOSURE

The present disclosure is in relation to a capsicum annuum extract comprising Carotenoids in a range of about 50% w/w to about 80% w/w.

In an embodiment of the present disclosure, the extract comprises Carotenoid Capsanthin in a range of about 35% w/w to about 60% w/w.

In another embodiment of the present disclosure, the Carotenoids comprises Capsanthin in a range of about 50% w/w to about 80% w/w of Carotenoids.

In still another embodiment of the present disclosure, the Capsanthin is trans-Capsanthin in a range of about 35% w/w to about 60% w/w and cis-Capsanthin in a range of about 0.1% w/w to about 5% w/w.

In still another embodiment of the present invention, the extract is of color value in a range of about 8 lakh CU to about 14 lakh CU.

In still another embodiment of the present disclosure, the extract is of red pigment value in a range of about 1000000 CU to about 1400000 CU

In still another embodiment of the present disclosure, the extract is in a form selected from a group comprising powder and solution, preferably powder.

In still another embodiment of the present invention, the extract is stable at temperature ranging from about 20° C. to about 30° C. for about period ranging from about 25 days to about 35 days.

The present disclosure is also in relation to a process for preparing Capsicum annuum extract comprising Carotenoids of about 50% w/w to about 80% w/w, said process comprising acts of,
a) subjecting purified paprika to supercritical fluid extraction to obtain deodorized paprika;
b) saponifying the deodorized paprika to obtain saponified products;
c) dissolving the saponified products in a solvent mixture comprising a organic solvent and water and separating the organic solvent layer and water layer;
d) washing the organic solvent layer with water to neutralize to about pH 7 to obtain the Capsicum annuum extract comprising Carotenoids of about 50% w/w to about 80% w/w.

In still another embodiment of the present disclosure, the purified paprika is obtained by a method comprising acts of,

a) subjecting de-seeded and powdered Capsicum annuum to solvent extraction using a solvent mixture to obtain a solvent extract;
b) de-solventising the solvent extract to obtain desolventised extract; and
c) isolating paprika from de-solventised extract by extraction using an aqueous alcohol and purifying the isolated paprika to obtain purified paprika.

In still another embodiment of the present disclosure, the super critical fluid extraction is carried out using carbon dioxide.

In still another embodiment of the present disclosure, the saponification is carried out using a base selected from a group comprising potassium hydroxide, sodium hydroxide, sodium carbonate, sodium bicarbonate, sodium methoxide and any combination thereof.

In still another embodiment of the present disclosure, the saponification is carried out at a temperature ranging from about 40° C. to about 70° C.

In still another embodiment of the present disclosure, the saponification is carried out for a period ranging from about 1 hour to about 24 hours.

In still another embodiment of the present disclosure, the solvent mixture comprises solvent selected from a group comprising acetone, pentane, hexane, heptane, ethylene dichloride, methylene dichloride, ethyl acetate, water and any combination thereof.

In still another embodiment of the present disclosure, the alcohol is selected from a group comprising methyl alcohol, ethyl alcohol, propyl alcohol, iso-propyl alcohol and butyl alcohol and combination thereof, preferably methyl alcohol.

The present disclosure is also in relation to a composition comprising Capsicum annuum extract comprising carotenoids of about of about 50% w/w to about 80% w/w along with one or more excipient.

In still another embodiment of the present disclosure, the excipients are in a range of about 65% w/w to about 80% w/w.

In still another embodiment of the present disclosure, the excipients are selected from a group comprising granulating agents, binding agents, lubricating agents, disintegrating agents, sweetening agents, glidants, anti-adherents, anti-static agents, surfactants, anti-oxidants, coating agents, coloring agents, flavouring agents, plasticizers, preservatives, suspending agents, emulsifying agents, speheronization agents and any combination thereof.

The present disclosure is also in relation to a method of using Capsicum annuum extract of the present disclosure or a composition comprising Capsicum annuum extract of the present disclosure, for enhancing HDL cholesterol in plasma, for cosmetic application, as a nutraceutical, as an antioxidant or as a health supplement said method comprising act of contacting the Capsicum annuum extract of the present disclosure or a composition of the present disclosure with a subject in need thereof.

In another embodiment of the present disclosure, the subject is an animal including human being.

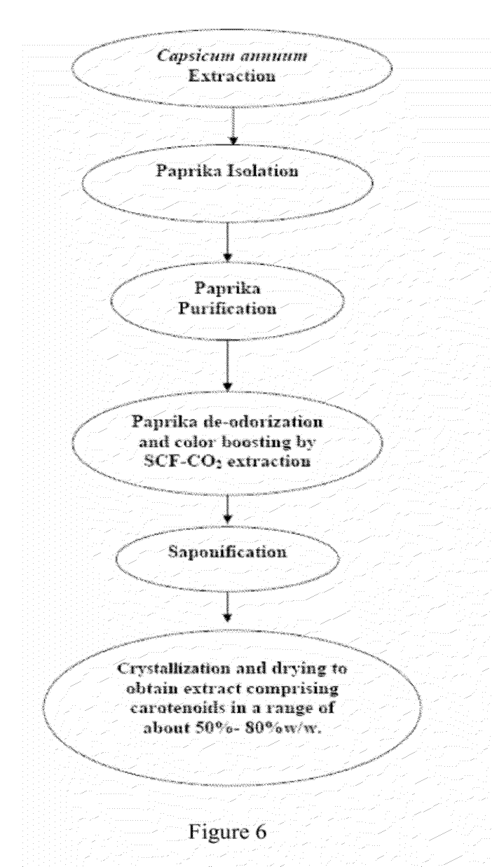

The present disclosure provides a Capsicum annuum extract comprising about 50% w/w to about 80% w/w of carotenoids. The Carotenoids is rich in Capsanthin with the content of the Capsanthin ranging from about 50% w/w to about 80% w/w of Carotenoids. In another embodiment of the present disclosure, the Capsicum annuum extract comprises Carotenoid, Capsanthin in a range of about 35% w/w to about 60% w/w. The Capsanthin is trans-Capsanthin in a range of about 35% w/w to about 60% w/w and cis-Capsanthin in a range of about 0.1% w/w to about 5% w/w of the Capsanthin content The Capsicum annuum extract is obtained by a simple and cost effective process as described in FIG. 6.

The present disclosure is also in relation to a composition comprising capsicum annuum extract comprising total carotenoids of about 50% w/w-80% w/w along with one or more pharmaceutically acceptable excipients. The composition can be utilized to enhance HDL cholesterol in plasma, for cosmetic applications, as nutraceutical, as antioxidant and as health supplement. The concentration of the extract and the excipients in the composition can be varied according to the need by a person skilled in the art. However, broadly the concentration can vary from about 65% w/w to about 80% w/w.

The details of method of extraction of carotenoids rich in Capsanthin, the details of the extract and their analysis are given below in the examples. However it should not be construed that the scope disclosure is limited to the examples.

### Example 1

The extraction has been carried out using Chillies of Indian—Byadagi variety.

### Step 1—Chilly Extraction:

100 kg Chillies are deseeded to get 65 kg pericarp which is powdered and used for extraction. The extraction is carried out using solvent mixture of hexane:acetone (70:30, 130 L) once. Subsequently the residue is further extracted five times with same solvent mixture (5×65 L). Extracted solvent layers are combined, filtered and desolventized to obtain 5 kg Chilly extract.

### Step 2—Paprika Isolation:

The 5 kg Chilly extract obtained in step 1 is subjected for liquid-liquid extraction. Separation of paprika and the capsaicin is carried out using 20 L of aqueous methanol (Sp.gravity 0.82 g/cc) as a solvent. The heavy phase layer is separated and passed through Wiped Film Evaporator after ensuring the complete removal of capsaicin (by HPLC analysis). 4.15 kg of the paprika product thus obtained is sand milled for maintaining the physical sediment below 1.5%

### Step 3—Paprika Purification:

4.15 kg paprika obtained in step 2 is mixed with MeOH (20 l) and is allowed stand for 1 hour to allow any sediments or insoluble residue to settle. The process is repeated twice to remove the insoluble residue. The clear methanol solution is separated and desolventized to get the purified product. Refined Paprika product obtained is 3.3 kg

Alternate process for single step paprika isolation and purification:

5 kg chili extract is subjected to methanol extraction (25 L, Sp. gravity 0.78-0.79 g/cc) using liquid-liquid extraction through gradient phase separation. The heavy phase is separated. This heavy phase is again subjected to liquid-liquid extraction using methanol (25 L) through gradient phase separation and heavy phase separated. The process is repeated five more times using methanol (5×25 L). The heavy phase isolated after the final extraction is passed through Wiped Film Evaporator after confirming the absence of pungency/residual capsaicin (by HPLC). The paprika thus obtained is sand milled for maintaining the physical sediment below 1.5%. Yield: 3.15 kg.

### Step 4—SCF-CO₂ for De-Odorization and Color Boosting:

3.3 kg of purified paprika obtained from step 3 is loaded into the super critical fluid extractor and CO₂ is pumped at a pressure of 140 bars and a temperature of 45° C. Process is continued for 1 hour for better deodorization and the extract part is collected. After 1 hour the pressure is increased to 400 bars and process is continued for 14 hours. The material residing in the SCF extractor is collected and the color value is analyzed using UV spectroscopy. The process is continued until the color value reached >320,000 cu. The extract collected is 1 kg.

### Step 5—Purification and Crystallization

The product obtained in step 4 (1 kg) is saponified using methanolic KOH (250 g of KOH in 4 L of MeOH). The mixture is stirred, maintaining the temperature in the range 50° C.-60° C. for 20 hours. The reaction is analyzed for the degree of saponification by HPLC and found to be above 95%. The solvent is evaporated completely under vacuum keeping the temperature below 55° C.

The product thus obtained is dissolved in ethyl acetate (7.5 L) and water (12.5 L) is added. The mixture is stirred for 2 hours and allowed to settle. After 4 hours layers are separated and ethyl acetate layer is collected. The aqueous layer is further extracted using ethyl acetate (2×5 L) and organic layers are combined. The aqueous layer is discarded.

The combined ethyl acetate portions are washed with water (25 L) until the pH becomes 7.0. The ethyl acetate portion is desolventised under vacuum to remove the residual solvent. The concentrate is suspended in hexane:acetone mixture (80:20, 2.8 L) and stirred for 1 hour. The mixture is filtered. The filtrate and the product are collected. The filtrate is kept for further crystallization and crystals obtained are combined and dried under vacuum. The product is re-crystallized from methanol (1.2 L). The crystals formed are filtered and dried under vacuum. The dried product is milled to get the 0.15 kg fine powder.

### Analytical Data of Final Product Obtained:

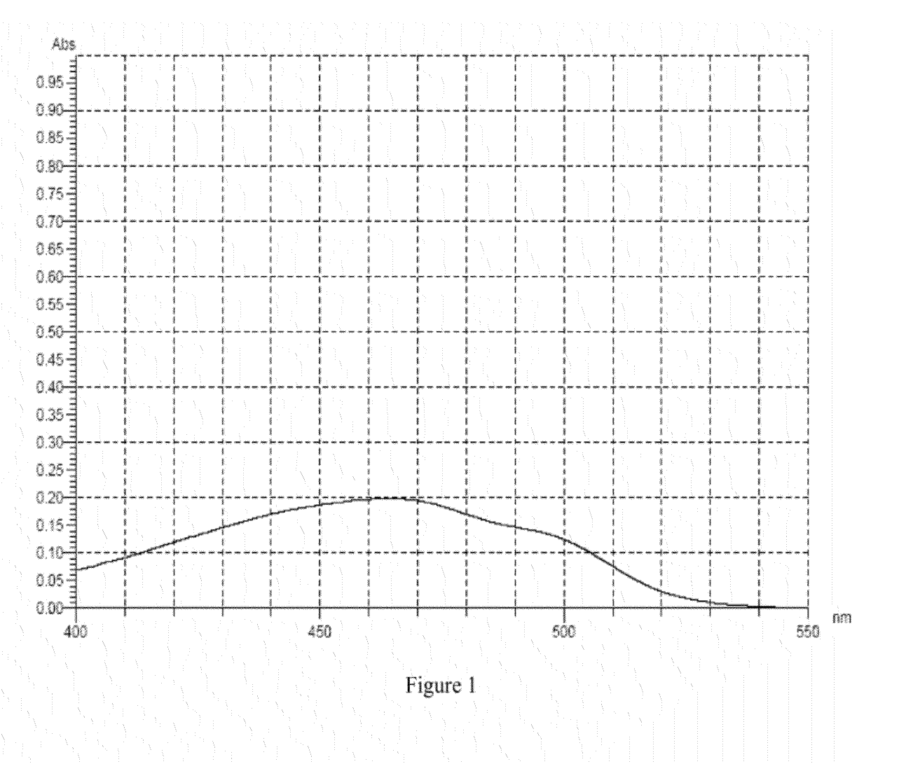

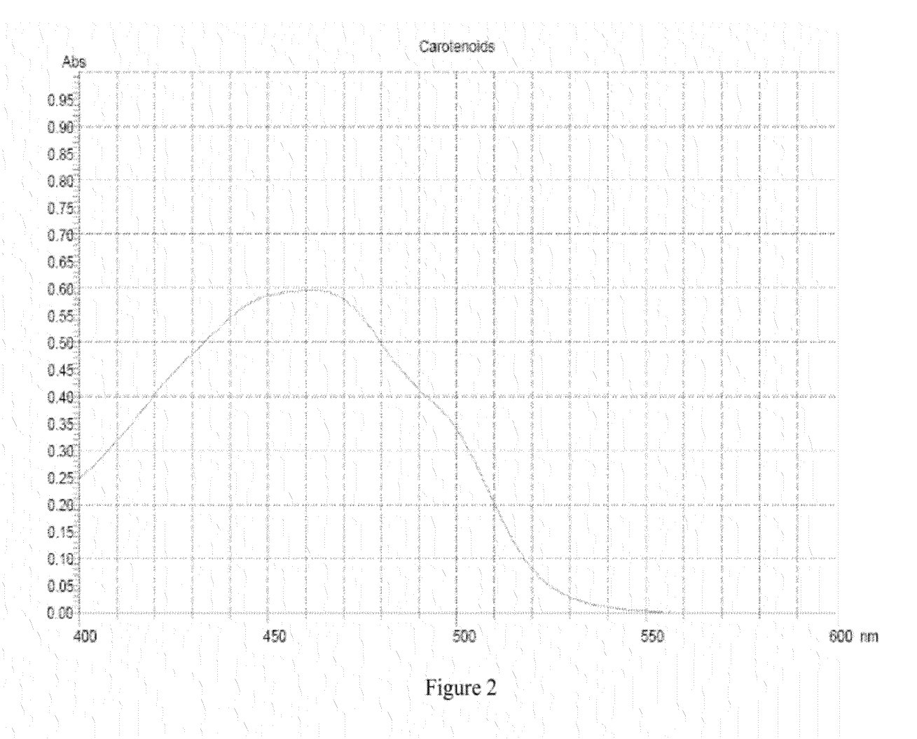

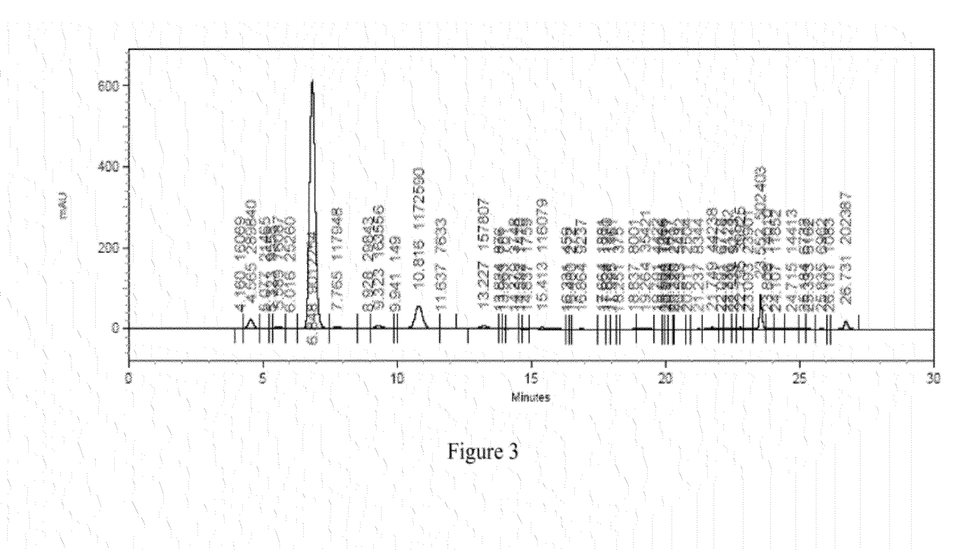

The product obtained from the above process is analyzed for its color value, A3 value, red pigment, total carotenoids content and trans-Capsanthin content accordingly the results analyzed from the FIGS. 1, 2 and 3; the analysis is as follows—

Color Value: 11.5 lakh
A3 value: 1.09

### Red Pigment: 99.55

Total Carotenoids content: 682 g/kg
trans-Capsanthin: 51%
Comparative Data from Literature with the Present Novel Extract:

| Parameter | Reported so far | Present extract |
|---|---|---|
| Total | 25-30% | 68% |
| carotenoid content | | (680 mg in 1 g of extract) |
| Capsanthin as | 40% | 75% of total Carotenoids |
| a % of total | | |
| carotenoids | | |
| Capsanthin | 32% | 51.6% |
| content w/w | | (516 mg in 1 g of extract) |
| trans-Capsanthin | Not available | 51% |
| content w/w | | |
| Cis-Capsanthin | Not available | 0.6% |
| content | | |
| Color value | 6 lakh CU | 11.5 Lakh CU |
| Red pigment | 60 Units | 99.5 Units |
| A3 value | Various | 1.08-1.09 Units |
| Color stability | Sensitive to heat | More stable to heat and |
| | and light, | light |
| | degrades very fast | |

### Discussion on the Analytical Data of the Present Novel Extract:

Carotenoids reported so far, are found to have capsanthin up to 40% of the total carotenoids. The present example has 680 mg of carotenoid per gram of the extract. It is found to have 75% of trans-capsanthin as a percentage in total carotenoids. The extract has total capsanthin of 51.6% w/w in which 51% w/w is all trans-capsanthin and 0.6% w/w is the cis-capsanthin. The product obtained directly from the process is in the form of deep red fine powder. The colour value (MSD 10 method) is found to be 11.5 Lakh CU; A3 value (MSD 10 method) of 1.08-1.09 Units. The red pigment value (MSD 10 method) for the product of the present disclosure is 99.5 Capsanthin purity is determined by reverse phase HPLC and total Carotenoids content is determined by UV/Vis spectroscopy. The product has much higher shelf life when compared to the reported carotenoids in the market. A study is conducted by keeping the product of the present disclosureat 120° C. for 48 hours in a hot air oven in an open Petri-dish. The colour value at the end of the experiment is decreased only to 11 lakh from 11.5 lakh. It may be noted that the color degradation is less than 5% indicated enhanced stability of the product of the present invention.

### Example 2

### From Chinese Paprika

### Step 1—Paprika Purification:

89.5 kg Chinese Paprika is mixed with MeOH (180 L) and is allowed stand for 1 hour to allow any sediments or insoluble residue to settle. The process is repeated twice to remove the insoluble residue. The clear methanol solution is separated and desolventized to get the purified product. Refined Paprika product obtained is 67.13 kg

### Step 2—SCF-CO₂ for De-Odorization and Color Boosting:

67.13 kg of purified paprika obtained from step 1 is loaded into the super critical fluid extractor and CO₂ is pumped at a pressure of 140 bars and a temperature of 45° C. Process is continued for 1 hour for better deodorization and the extract part is collected. After 1 hour the pressure is increased to 400 bars and process is continued for 14 hours. The material residing in the SCF extractor is collected and the color value is analyzed using UV spectroscopy. The process is continued until the color value reached >300,000 cu. The extract collected is 27.3 kg.

### Step 3—Purification and Crystallization:

The product obtained in step 2 (27.3 kg) is saponified using methanolic KOH (7 Kg of KOH in 110 L of MeOH). The mixture is stirred, maintaining the temperature in the range 50° C.-60° C. for 20 hours. The reaction is analyzed for the degree of saponification by HPLC and found to be above 95%. The solvent is evaporated completely under vacuum keeping the temperature below 55° C.

The product thus obtained is dissolved in ethyl acetate (225 L) and water (300 L) is added. The mixture is stirred for 2 hours and allowed to settle. After 4 hours layers were separated and ethyl acetate layer is collected. The aqueous layer is further extracted using ethyl acetate (2×215 L) and organic layers were combined. The aqueous layer is discarded.

The combined ethyl acetate portions were washed with water (650 L) until the pH becomes 7.0. The ethyl acetate portion is desolventized under vacuum to remove the residual solvent. The concentrate is suspended in hexane:acetone mixture (80:20, 100 L) and stirred for 1 hour. The mixture is filtered. The filtrate and the product were collected. The filtrate is kept for further crystallization and crystals obtained were combined and dried under vacuum. The product is re-crystallized from methanol (15 L). The crystals formed were filtered and dried under vacuum. The dried product is milled to get the 2.5 kg fine powder.

### Analytical Data of Final Product Obtained:

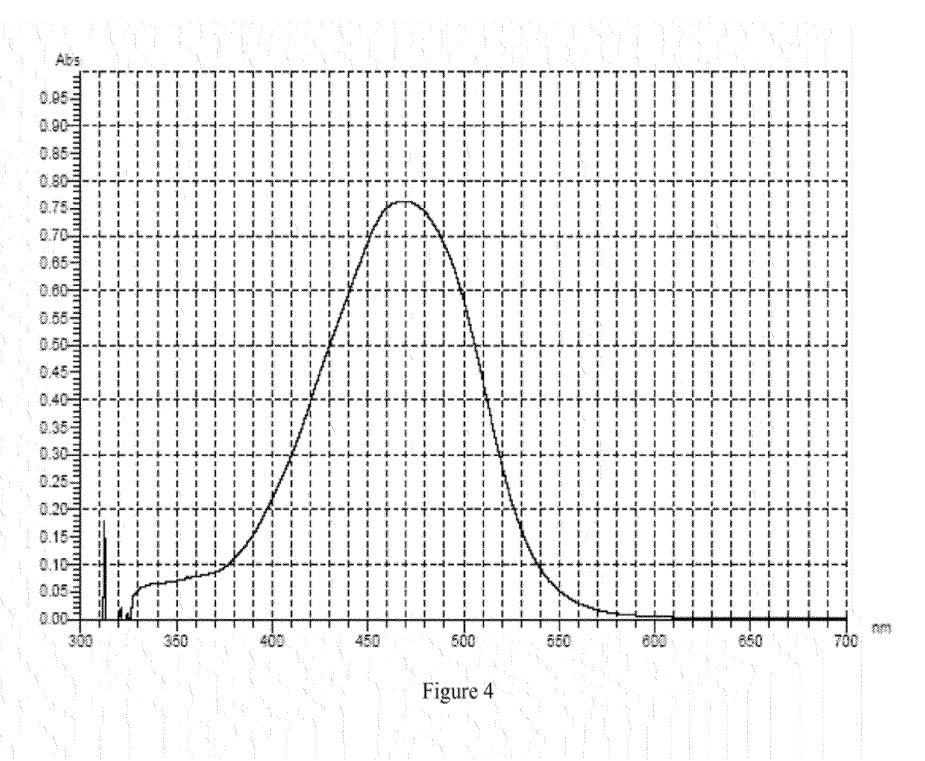

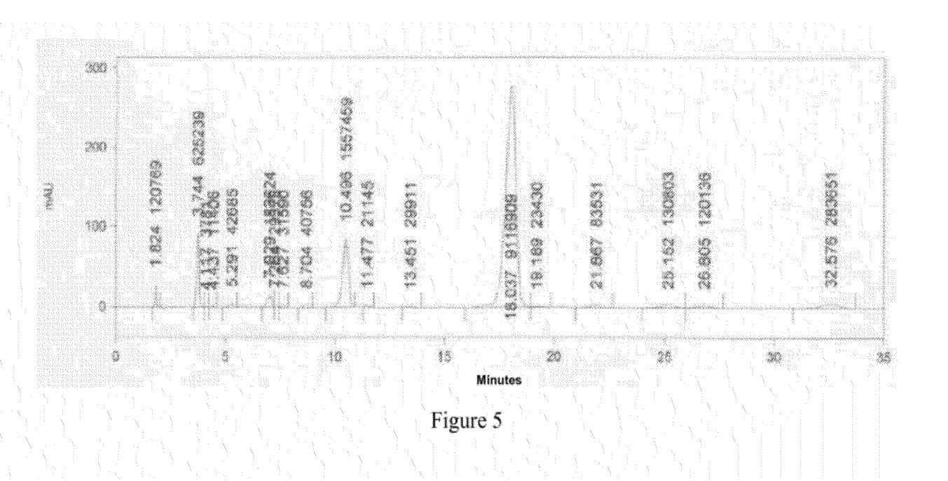

The FIG. 4 provides the information on the Color Value, A3 value and Red Pigment. Accordingly the values are as follows. The HPLC analysis as provided in FIG. 5 informs about the content of trans-Capsanthin.

Color Value: 11.75 lakh
A3 value: 1.05

### Red Pigment: 87.98

Total Carotenoids content: 710 g/kg
trans-Capsanthin: 51.9%

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

We claim:

1. A Capsicum annuum extract comprising trans-Capsanthin ranging from about 35% w/w to about 60% w/w and cis-Capsanthin ranging from about 0.1% w/w to about 5% w/w, wherein the extract has color value ranging from about 800000 CU to about 1400000 CU.

2. The Capsicum annuum extract as claimed in claim 1, wherein the extract is of red pigment value ranging from about 87.98 units to about 99.5 units.

3. The Capsicum annuum extract as claimed in claim 1, wherein the extract is in a form selected from a group comprising powder and solution, preferably powder.

4. The Capsicum annuum extract as claimed in claim 1, wherein the extract is stable at temperature ranging from about 20° C. to about 30° C. for time period ranging from about 25 days to about 35 days.

5. A process for preparing Capsicum annuum extract comprising trans-Capsanthin in a range of about 35% w/w to about 60% w/w and cis-Capsanthin in a range of about 0.1% w/w to about 5% w/w, wherein the extract has color value ranging from about 800000 CU to about 1400000 CU, said process comprising acts of,
a) subjecting purified paprika to supercritical fluid extraction to obtain deodorized paprika;
b) saponifying the deodorized paprika to obtain saponified products;
c) dissolving the saponified products in a solvent mixture comprising a organic solvent and water, and separating the organic solvent layer; and
d) washing the organic solvent layer and neutralizing to about pH 7, and obtaining the Capsicum annuum extract comprising trans-Capsanthin in a range of about 35% w/w to about 60% w/w and cis-Capsanthin in a range of about 0.1% w/w to about 5% w/w, wherein the extract has color value ranging from about 800000 CU to about 1400000 CU.

6. The process as claimed in claim 5, wherein the purified paprika is obtained by a method comprising acts of,
a) subjecting de-seeded and powdered Capsicum annuum to solvent extraction using a solvent mixture to obtain a solvent extract;
b) de-solventising the solvent extract to obtain desolventised extract; and
c) isolating paprika from de-solventised extract by extraction using an aqueous alcohol and purifying the isolated paprika to obtain the purified paprika.

7. The process as claimed in claim 5, wherein the super critical fluid extraction is carried out using carbon dioxide.

8. The process as claimed in claim 5, wherein the saponification is carried out using base selected from a group comprising potassium hydroxide, sodium hydroxide, sodium carbonate, sodium bicarbonate, and sodium methoxide or any combination thereof.

9. The process as claimed in claim 5, wherein the saponification is carried out at a temperature ranging from about 40° C. to about 70° C.

10. The process as claimed in claim 5, wherein the saponification is carried out for a time period ranging from about 1 hour to about 24 hours.

11. The process as claimed in claim 5, wherein the solvent mixture comprises solvent selected from a group comprising acetone, pentane, hexane, heptane, ethylene dichloride, methylene dichloride, ethyl acetate, and water or any combination thereof.

12. The process as claimed in claim 5, wherein the alcohol is selected from a group comprising methyl alcohol, ethyl alcohol, propyl alcohol, iso-propyl alcohol and butyl alcohol or any combination thereof, preferably methyl alcohol.

13. A composition comprising Capsicum annuum extract comprising trans-Capsanthin ranging from about 35% w/w to about 60% w/w and cis-Capsanthin ranging from about 0.1% w/w to about 5% w/w, wherein the extract has color value ranging from about 800000 CU to about 1400000 CU along with one or more excipient.

14. The composition as claimed in claim 13, wherein the excipients are in a range of about 65% w/w to about 80% w/w.

15. The composition as claimed in claim 13, wherein the excipients are selected from a group comprising granulating agents, binding agents, lubricating agents, disintegrating agents, sweetening agents, glidants, anti-adherents, anti-static agents, surfactants, anti-oxidants, coating agents, coloring agents, flavouring agents, plasticizers, preservatives, suspending agents, emulsifying agents, and speheronization agents or any combination thereof.

16. A method of using Capsicum annuum extract as claimed in claim 1, for enhancing HDL cholesterol in plasma, for cosmetic application, as a nutraceutical, as an antioxidant or as a health supplement said method comprising act of contacting the extract or the composition with a subject in need thereof.

17. The method as claimed in claim 16, wherein the subject is an animal including human being.

18-21. (canceled)

22. A method of using the composition as claimed in claim 13 for enhancing HDL cholesterol in plasma, for cosmetic application, as a nutraceutical, as an antioxidant or as a health supplement said method comprising act of contacting the extract or the composition with a subject in need thereof.
